(19) United States
(12) Patent Application Publication
(10) Pub. No.: US 2015/0328145 A1
(43) Pub. Date: Nov. 19, 2015

(54) TOPICAL OPHTHALMOLOGICAL PHARMACEUTICAL COMPOSITION CONTAINING REGORAFENIB

(71) Applicants: Petra HEINRICH-KELDENICH, Wuppertal (DE); BAYER HEALTHCARE LLC, Whippany, NJ (US)

(72) Inventors: Michael BOTTGER, Wuppertal (DE); Georges VON DEGENFELD, Leverkusen (DE); Julia FREUNDLIEB, Dortmund (DE); Claudia HIRTH-DIETRICH, Wuppertal (DE); Joerg KELDENICH, Wuppertal (DE); Jurgen KLAR, Wuppertal (DE); Uwe MUENSTER, Wulfrath (DE); Andreas OHM, Neuss (DE); Annett RICHTER, Berlin (DE); Bernd RIEDL, Wuppertal (DE)

(73) Assignee: BAYER HEALTHCARE LLC, Whippany, NJ (US)

(21) Appl. No.: 14/653,317

(22) PCT Filed: Dec. 21, 2013
(86) PCT No.: PCT/US2013/077358

§ 371 Date: Jun. 18, 2015

(30) Foreign Application Priority Data
Dec. 21, 2012 (EP) 12198892.7

(51) Int. Cl. A61K 9/00; A61K 47/14; A61K 47/06; A61K 31/44

(52) U.S. Cl. CPC A61K 9/0014; A61K 9/0048; A61K 31/44; A61K 47/14; A61K 47/06

(57) ABSTRACT

The present invention relates to topical ophthalmological pharmaceutical compositions containing regorafenib, a hydrate, solvate or pharmaceutically acceptable salt thereof or a polymorph thereof but without hydrophobic silica and its process of preparation and its use for treating ophthalmological disorders.

The present invention relates to topical ophthalmological pharmaceutical compositions containing regorafenib, a hydrate, solvate or pharmaceutically acceptable salt thereof or a polymorph thereof but without hydrophobic silica and its process of preparation and its use for treating ophthalmological disorders.

Regorafenib which is 4{4-[3-(4-chloro-3-trifluoromethylphenyl)-ureido]-3-fluorophenoxy}-pyridine-2-carboxylic acid methylamide, a compound of formula (I)

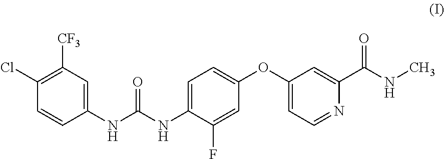 [SMILES: CNC(=O)c1cc(Oc2ccc(NC(=O)Nc3ccc(Cl)c(C(F)(F)F)c3)c(F)c2)ccn1]

is a potent anti-cancer and anti-angiogenic agent that possesses various activities including inhibitory activity on the VEGFR, PDGFR, raf, p38, and/or flt-3 kinase signalling molecules and it can be used in treating various diseases and conditions like hyper-proliferative disorders such as cancers, tumors, lymphomas, sarcomas and leukemias as described in WO 2005/009961. Furthermore salts of the compound of formula (I) such as its hydrochloride, mesylate and phenylsulfonate are mentioned in WO 05/009961. The monohydrate of the compound of formula (I) is mentioned in WO 08/043446.

Age-related macular degeneration (AMD) is a leading cause of blindness in the elderly population and is recognized as dry and wet AMD (Expert Opin. Ther. Patents (2010), 20(1), 103-11). The dry, or nonexudative, form involves both atrophic and hypertrophic changes of the retinal pigment epithelium (RPE). The dry form is characterized by macular drusen which are pigmented areas containing dead cells and metabolic products that distort the retina and eventually cause loss of acute vision. Patients with nonexudative AMD (dry form) can progress to the wet, or exudative or neovascular, AMD, in which pathologic choroidal neovascular membranes (CNVM) develop under the retina, leak fluid and blood, and, ultimately, cause a centrally blinding disciform scar over a relatively short time frame if left untreated. Choroidal neovascularization (CNV), the growth of new blood vessels from the choroid capillary network across the Bruch's membrane/RPE interface into the neural retina, results in retinal detachment, subretinal and intraretinal edema, and scarring.

Access to the choroid which is between the sclera and the retina other than via the blood is difficult. The eye is composed of three major anatomic compartments, the anterior chamber, posterior chamber, and vitreous cavity, that have limited physiological interaction with each other. The retina is located in the back of the vitreous cavity, and is protected from the outside by the sclera which is the white, tough, impermeable wall of the eye. Choroidal blood flow is the usual method of carrying substances to the choroid and requires e.g. oral or intravenous administration of the drug. Most drugs cannot be delivered to the choroid by eye drops or a depot in vicinity to the eye. Some drugs have been delivered to the retina and thus to the choroid by injection into the vitreous chamber of the eye. The treatment of posterior eye diseases (back of the eye) by easily applicable topical eye formulations like eye drops is still an unsolved problem.

VEGF (vascular endothelial growth factor) is a key growth factor in the development of normal blood vessels as well as the development of vessels in tumors and other tissues undergoing abnormal angiogenesis and appears to play a central role in the pathogenesis of CNV formation (Expert Opin. Ther. Patents (2010), 20(1), 103-118, Expert Opin. Ther. Patents (2009), 18(10), 1573-1580, J. Clin. Invest. (2010), 120(9), 3033-3041, J. Cell. Physiol. (2008), 216, 29-37, New Engl. J. Med. 2006, 355, 1474-1485, WO 2010/127029, WO 2007/064752). Drugs which block the effects of VEGF are described for treating wet AMD such as aptamers like pegaptanib (New Engl. J. Med. 2004, 351, 2805-2816), or VEGF antibodies like ranibizumab (New Engl. J. Med. 2006, 355, 1419-1431) or bevacizumab (Ophthalmology, 2006, 113, 363-372). However, said drugs have to be administered intravitreally by injection into the eye. Sorafenib, a VEGF inhibitor as well, is described for treating CNV by oral administration (Clinical and Experimental Ophthalmology, 2010, 38, 718-726). Pazopanib, a VEGF inhibitor as well, is described for treating AMD by topical administration of eye drops containing an aqueous solution of Pazopanib (WO 2011/009016). WO 2006/133411 describes compounds for the treatment of CNV by topical administration of liposomal formulations. WO 2007/076358, US2006257487 describe aqueous ophthalmological formulations for topical administration. WO 2008/27341 describes emulsions for topical administration to the eye.

It is general expert knowledge that usually topical eye drops do not deliver therapeutic levels of drug molecules to the target tissues present at the back of the eye in order to treat posterior eye diseases (U. B. Kompella and H. F. Edelhauser, “Drug Product Development for the Back of the Eye”, aapspress Springer, 2011, page 449).

Despite the progress described in the art there remains a need for improved medicines for the treatment of ophthalmological disorders like AMD. In particular, there remains a need for topical ophthalmological pharmaceutical compositions like eye drops which can be administered easily and therefore would increase the patient's compliance. Furthermore there is still the need of applicable topical ophthalmological pharmaceutical compositions for compounds having for example a low solubility which cannot be formulated in a simple solution, emulsion, as a complex or in a liposomal formulation. The topical ophthalmological pharmaceutical composition has to provide a concentration of the active agent in the eye which is sufficient for an effective therapy. This is dependent on the solubility and the release behavior of the active agent. In the case of a liquid formulation the dissolution properties and chemical stability of the active agent are of importance. In order to support a high compliance the topical ophthalmological pharmaceutical composition should not have to be taken in more than 5 times a day, the less the better. Type and amount of the excipients in combination with the process of preparation of the pharmaceutical composition are essential for release properties, bioavailability of the active agent in the eye, in particular in the back of the eye (e.g. in the area of the retina, Bruch's membrane and choroid), stability, compatibility, efficacy and the industrial applicability of the manufacturing process for the topical ophthalmological pharmaceutical composition.

The problem to be solved by the present invention is to provide a topical ophthalmological pharmaceutical composition comprising regorafenib as active agent which has a sufficient stability and compatibility and which achieves an effective concentration of regorafenib in the eye, in particular in the back of the eye for the treatment of ophthalmological disorders with sufficient efficacy by avoiding an intravenous or oral administration or injection into or close to the eye (e.g. intravitreal or other injections).

Another problem to be solved by the present invention is to provide a topical ophthalmological pharmaceutical composition for the treatment of a posterior eye disease.

Regorafenib monohydrate has a limited solubility profile. The thermodynamic solubility of regorafenib monohydrate in different solvents is shown in table 1:

TABLE 1

| Solvent | Solubility (mg/ml) |
|---|---|
| Water | <0.1 |
| Light liquid paraffin | <0.1 |
| Ethanol | 6.4 |
| Polyethylenglycol (PEG) 400 | 67.3 |
| HPβ-Cyclodextrin/water (10:90) | <0.1 |
| PEG 400/water (30:70) | 0.27 |
| Oleoylpolyethylenglycol glycerides | 3.6 |

Surprisingly the pharmaceutical composition according to the invention provides by topical administration a sufficient amount of the active agent into the eye which is effective for treating ophthalmological disorders. In particular, the pharmaceutical composition according to the invention provides the active agent in a sufficient amount into the back of the eye, i.e. that the pharmaceutical composition according to the invention effects the transportation of the active agent from the front of the eye to the back of the eye. Furthermore the pharmaceutical composition according to the invention has a sufficient stability without any meaningful degradation of the active agent and is compatible with the eye.

The present invention pertains to a topical ophthalmological pharmaceutical composition comprising regorafenib, the compound of the formula (I),

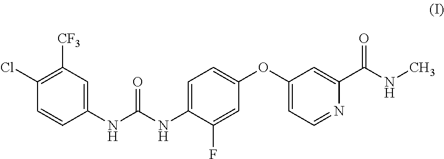 [SMILES: CNC(=O)c1cc(Oc2ccc(NC(=O)Nc3ccc(Cl)c(C(F)(F)F)c3)c(F)c2)ccn1]

a hydrate, solvate or pharmaceutically acceptable salt of regorafenib, or a polymorph thereof and at least one pharmaceutically acceptable vehicle and optionally at least one pharmaceutically acceptable excipient wherein the composition does not contain hydrophobic silica.

Preference is given to a topical ophthalmological pharmaceutical composition comprising regorafenib, a hydrate, solvate or pharmaceutically acceptable salt of regorafenib or a polymorph thereof as active agent and at least one pharmaceutically acceptable vehicle and optionally at least one pharmaceutically acceptable excipient wherein the composition is a suspension comprising the active agent suspended in the applicable pharmaceutically acceptable vehicle and wherein the composition does not contain hydrophobic silica.

A pharmaceutically acceptable vehicle or excipient is any vehicle or excipient which is relatively non-toxic and innocuous to a patient at concentrations consistent with effective activity of the active agent so that any side effects ascribable to the vehicle or excipient do not vitiate the beneficial effects of the active agent.

The term “the compound of formula (I)” or “regorafenib” refer to 4-{4-[({[4-chloro-3-(trifluoromethyl)phenyl]amino]carbonyl)amino}-3-fluorophenoxy}-N-methylpyridine-2-carboxamide as depicted in formula (I).

The term “compound of the invention” or “active agent” refer to regorafenib, a hydrate, solvate or pharmaceutically acceptable salt of regorafenib, or a polymorph thereof.

Solvates for the purposes of the invention are those forms of the compounds or their salts where solvent molecules form a stoichiometric complex in the solid state and include, but are not limited to for example ethanol and methanol.

Hydrates are a specific form of solvates, where the solvent molecule is water. Hydrates of the compounds of the invention or their salts are stoichiometric compositions of the compounds or salts with water, such as, for example, hemi-, mono- or dihydrates. Preference is given to the monohydrate of regorafenib.

Salts for the purposes of the present invention are preferably pharmaceutically acceptable salts of the compounds according to the invention. Suitable pharmaceutically acceptable salts are well known to those skilled in the art and include salts of inorganic and organic acids, such as hydrochloric acid, hydrobromic acid, sulfuric acid, phosphoric acid, methanesulphonic acid, trifluoromethanesulfonic acid, benzenesulfonic acid, p-toluenesulfonic acid (tosylate salt), 1-naphthalenesulfonic acid, 2-naphthalenesulfonic acid, acetic acid, trifluoroacetic acid, malic acid, tartaric acid, citric acid, lactic acid, oxalic acid, succinic acid, fumaric acid, maleic acid, benzoic acid, salicylic acid, phenylacetic acid, and mandelic acid. In addition, pharmaceutically acceptable salts include salts of inorganic bases, such as salts containing alkaline cations (e.g., Li⁺ Na⁺ or K⁺, alkaline earth cations (e.g., Mg⁺², Ca⁺² or Ba⁺²), the ammonium cation, as well as acid salts of organic bases, including aliphatic and aromatic substituted ammonium, and quaternary ammonium cations, such as those arising from protonation or peralkylation of triethylamine, N,N-diethylamine, N,N-dicyclohexylamine, lysine, pyridine, N,N-dimethylaminopyridine (DMAP), 1,4-diazabiclo[2.2.2]octane (DABCO), 1,5-diazabicyclo[4.3.0]non-5-ene (DBN) and 1,8-diazabicyclo[5.4.0]undec-7-ene (DBU). Preference is given to the hydrochloride, mesylate or phenylsulfonate salt of regorafenib.

Preferred are regorafenib and the monohydrate of regorafenib, most preferred is regorafenib monohydrate as compounds of the present invention.

Due to the low solubility of regorafenib, in particular of regorafenib monohydrate (see table 1) standard solutions are not applicable. Also solutions containing tolerable amounts of emulsifiers, solubilising agents, complex forming excipients etc. are not available to provide for example sufficient stability of regorafenib.

The topical ophthalmological pharmaceutical composition according to the invention comprises the compound of the invention, preferably regorafenib, more preferably regorafenib monohydrate in a solid form, preferably in a crystalline form, more preferably in a microcrystalline form.

Micronization can be achieved by standard milling methods, preferably by air jet milling, known to a skilled person. The microcrystalline form can have a mean particle size of from 0.5 to 10 μm, preferably from 1 to 6 μm, more preferably from 1 to 3 μM. The indicated particle size is the mean of the particle size distribution measured by laser diffraction known to a skilled person (measuring device: HELOS, Sympatec).

The minimum concentration of the compound of the invention, preferably regorafenib, more preferably regorafenib monohydrate in the topical ophthalmological pharmaceutical composition is 0.01%, preferably 0.2% by weight of the total amount of the composition. The maximum concentration of the compound of the invention, preferably regorafenib, more preferably regorafenib monohydrate in the topical ophthalmological pharmaceutical composition is 10%, preferably 6%, more preferably 5%, most preferably 4% by weight of the total amount of the composition.

Preference is given to a concentration of the compound of the present invention in the pharmaceutical composition from 0.1 to 100 mg/ml, preferably from 1 to 50 mg/ml, more preferably from 2 to 40 mg/ml.

Particular preference is given to a concentration of regorafenib in the pharmaceutical composition from 0.1 to 100 mg/ml, preferably from 1 to 50 mg/ml, more preferably from 2 to 40 mg/ml.

Particular preference is given to a pharmaceutical composition resulting from addition of regorafenib monohydrate in amounts from 0.1 to 100 mg/ml, preferably from 1 to 50 mg/ml, more preferably from 2 to 40 mg/ml.

The topical ophthalmological pharmaceutical composition according to the invention includes but is not limited to eye drops, gels, ointments, dispersions or suspensions.

Preference is given to a topical ophthalmological pharmaceutical composition which is a suspension.

The compound of the invention, preferably regorafenib, more preferably regorafenib monohydrate is used preferably in a micronized form.

Micronization can be achieved by standard milling methods, preferably by air jet milling, known to a skilled person. The micronized form can have a mean particle size of from 0.5 to 10 μm, preferably from 1 to 6 μm, more preferably from 2 to 3 μm. The indicated particle size is the mean of the particle size distribution measured by laser diffraction known to a skilled person (measuring device: HELOS, Sympatec).

One embodiment of the present invention is a topical ophthalmological pharmaceutical composition which is a suspension comprising the compound of the invention, preferably regorafenib, more preferably regorafenib monohydrate in a solid form, preferably in a crystalline form, more preferably in a microfine crystalline form suspended in an applicable pharmaceutically acceptable vehicle, and optionally further comprising one or more pharmaceutically acceptable excipients wherein the composition does not contain hydrophobic silica.

Preference is given to a suspension based on a non-aqueous vehicle, more preferably to a suspension based on a hydrophobic vehicle.

Suitable pharmaceutically acceptable vehicles according to the present invention include but are not limited to oleoyl polyethyleneglycol gylcerides, linoleoyl polyethyleneglycol gylcerides, lauroyl polyethyleneglycol gylcerides, hydrocarbon vehicles like liquid paraffin (Paraffinum liquidum, mineral oil), light liquid paraffin (low viscosity paraffin, Paraffinum perliquidum, light mineral oil), soft paraffin (vaseline), hard paraffin, vegetable fatty oils like castor oil, peanut oil or sesame oil, synthetic fatty oils like middle chain trigylcerides (MCT, triglycerides with saturated fatty acids, preferably octanoic and decanoic acid), isopropyl myristate, caprylocaproyl macrogol-8 glyceride, caprylocaproyl polyoxyl-8 glycerides, wool alcohols like cetylstearylalcohols, wool fat, glycerol, propylene glycol, propylene glycol diesters of caprylic/capric acid, polyethyleneglycols (PEG), water like an aqueous isotonic sodium chloride solution or a mixture of thereof.

Preference is given to non-aqueous pharmaceutically acceptable vehicles which include but are not limited to middle chain trigylcerides (MCT, triglycerides with saturated fatty acids, preferably octanoic and decanoic acid, isopropyl myristate, caprylocaproyl macrogol-8 glyceride, caprylocaproyl polyoxyl-8 glycerides, oleoyl polyethyleneglycol glycerides, oleoyl macrogol-6 glycerides (Labrafil M 1944 CS), linoleoyl macrogol-6 glycerides (Labrafil M2125 CS=linoleoyl polyoxyl-6 glycerides), lauroyl macrogol-6 glycerides (Labrafil M 2130 CS=lauroyl polyoxyl-6 glycerides)), hydrocarbon vehicles, fatty oils like castor oil or a mixture of thereof. Most preferably hydrophobic vehicles are used like hydrocarbon vehicles which include but are not limited to liquid paraffin or light liquid paraffin or a mixture thereof.

Very surprisingly the pharmaceutical composition according to the present invention comprising a lipophilic (or hydrophobic) vehicle like liquid or light liquid paraffin provides by topical administration a sufficient amount of the active agent into the eye which is effective for treating ophthalmological disorders, although the solubility of regorafenib monohydrate in lipophilic (or hydrophobic) vehicles is very low.

The pharmaceutically acceptable vehicle is the basis of the topical ophthalmological pharmaceutical composition according to the present invention and is present in the composition in a minimum concentration of 75%, preferably 80%, more preferably 85% and in a maximum concentration of 99.9%, preferably 99%, more preferably 98% by weight of the total amount of the composition.

The pharmaceutical composition according to the present invention may have different viscosities, so that in principle a range from low-viscosity system to pastes is conceivable. Preference is given to fluid systems which include low-viscosity and also higher-viscosity systems as long as they still flow under their own weight.

Suitable further pharmaceutically acceptable excipients used in the topical ophthalmological pharmaceutical composition according to the present invention include but are not limited to surfactants, polymer based carriers like gelling agents, organic co-solvents, pH active components, osmotic active components and preservatives. Preferably the composition does not contain a stabilizer.

Suitable surfactants used in the topical ophthalmological pharmaceutical composition according to the present invention include but are not limited to lipids such as phospholipids, phosphatidylcholines, lecithin, cardiolipins, fatty acids, phosphatidylethanolamines, phosphatides, tyloxapol, polyethylenglycols and derivatives like PEG 400, PEG 1500, PEG 2000, poloxamer 407, poloxamer 188, polysorbate 80, polysorbate 20, sorbitan laurate, sorbitan stearate, sorbitan palmitate or a mixture thereof, preferably polysorbate 80.

Suitable polymer base carriers like gelling agents used in the topical ophthalmological pharmaceutical composition according to the present invention include but are not limited to cellulose, hydroxypropylmethylcellulose (HPMC), hydroxypropylcellulose (HPC), carboxymethyl cellulose (CMC), methylcellulose (MC), hydroxyethylcellulose (HEC), amylase and derivatives, amylopectins and derivatives, dextran and derivatives, polyvinylpyrrolidone (PVP), polyvinyl alcohol (PVA), and acrylic polymers such as derivatives of polyacrylic or polymethacrylic acid like HEMA, carbopol and derivatives of the before mentioned or a mixture thereof.

Suitable organic co-solvents used in the pharmaceutical composition according to the invention include but are not limited to ethylene glycol, propylene glycol, N-methyl pyrrolidone, 2-pyrrolidone, 3-pyrrolidinol, 1,4-butanediol, dimethylglycol monomethylether, diethyleneglycol monomethylether, solketal, glycerol, polyethylene glycol, polypropylene glycol.

Suitable pH active components such as buffering agents or pH-adjusting agents used in the pharmaceutical composition according to the invention include but are not limited to disodium phosphate, monosodium phosphate, boric acid, sodium borate, sodium citrate, hydrochloric acid, sodium hydroxide.

The pH active components are chosen based on the target pH for the composition which generally ranges from pH 4-9.

Suitable osmotic active components used in the pharmaceutical composition according to the invention include but are not limited to sodium chloride, mannitol, glycerol.

Preservatives used in the pharmaceutical composition according to the invention include but are not limited to benzalkonium chloride, alkyldimethylbenzylammonium chloride, cetrimide, cetylpyridinium chloride, benzododecinium bromide, benzethonium chloride, thiomersal, chlorobutanol, benzyl alcohol, phenoxethanol, phenylethyl alcohol, sorbic acid, methyl and propyl parabens, chlorhexidine digluconate, EDTA or mixtures thereof.

Gelling agents, pH active agents and osmotic active agents are preferably used in the case of an aqueous pharmaceutically acceptable vehicle.

The amount of the suitable further pharmaceutically acceptable excipient in the suspension according to the present invention can be from 0.1 to 15%, preferably from 0.5 to 10%, more preferably from 1 to 5% by the total weight of the suspension.

Preferably the amount of hydroxypropylmethylcellulose in the suspension according to the present invention can be from 0.05 to 15%, preferably from 0.1 to 10%, more preferably from 1 to 5% by the total weight of the suspension.

Preferably the amount of polysorbate 80 in the suspension according to the present invention can be from 0.05 to 10%, preferably from 0.1 to 7%, more preferably from 0.5 to 4% by the total weight of the suspension.

The pharmaceutical composition according to the present invention does not contain hydrophobic silicas, preferably does not contain stabilizers including colloidal silica, hydrophilic or hydrophobic silicas.

Hydrophobic silicas are silicas which are not wetted by water; this means that they float on the water surface. They are produced by mixing hydrophilic silica with silanes (halosilanes, alkoxysilanes, silazanes, siloxanes). This entails silanol groups being alkylated by alkyl groups preferably having one up to 18 carbon atoms, particularly preferably having one up to 8 carbon atoms, very particularly preferably having one up to 4 carbon atoms, especially by methyl groups. Examples of silanes used in the production of hydrophobic silicas are hexamethyldisilazane or, preferably, dimethyldichlorosilane. The appropriate hydrophobic silicas may be derived from precipitated, colloidal, precompacted or pyrogenic silicas, with preference for pyrogenic silicas. For example, reaction of a hydrophilic silica with dimethyldichlorosilane results in hydrophobic Aerosil having the proprietary name Aerosil® R 972; this has a degree of methylation of 66% to 75% (determined by titration of the remaining silanol groups).

Preference is given to a topical ophthalmological pharmaceutical composition comprising crystalline regorafenib monohydrate, more preferably microcrystalline regorafenib monohydrate in a concentration of for example 0.01 to 10%, more preferably 0.2 to 5% weight of the total amount of the composition suspended in a pharmaceutically acceptable vehicle selected from the group comprising liquid paraffin, light liquid paraffin or a mixture thereof wherein the composition does not contain hydrophobic silica.

Preference is also given to a topical ophthalmological pharmaceutical composition comprising crystalline regorafenib monohydrate, more preferably microfine crystalline regorafenib monohydrate in a concentration of for example 0.1 to 10%, more preferably 0.2 to 5% weight of the total amount of the composition suspended in oleoyl polyethyleneglycol glyceride as pharmaceutically acceptable vehicle wherein the composition does not contain hydrophobic silica.

The total amount of the active agent to be administered via the topical route into the eye using the pharmaceutical composition of the present invention will generally range from about 0.01 to 50 mg, preferably 0.02 to 10 mg, more preferably 0.05 to 5 mg per administration and per eye. Based upon standard laboratory techniques known to evaluate compounds useful for the treatment of ophthalmological disorders, by standard pharmacological assays for the determination of treatment of the conditions identified above in mammals, and by comparison of these results with the results of known medicaments that are used to treat these conditions, the effective dosage of the pharmaceutical compositions of this invention can readily be determined by those skilled in the art. The amount of the administered active ingredient can vary widely according to such considerations as the particular compound and dosage unit employed, the mode and time of administration, the period of treatment, the age, sex, and general condition of the patient treated, the nature and extent of the condition treated, the rate of drug metabolism and excretion, the potential drug combinations and drug-drug interactions, and the like.

The pharmaceutical composition according to the invention is administered one or more, preferably up to 5, more preferably up to 3 times per day.

The typical method of administration of the pharmaceutical composition according to the invention is the topical delivery into the eye.

Nevertheless, it may in some cases be advantageous to deviate from the amounts specified, depending on individual response to the active ingredient, type of preparation and time or interval over which the administration is effected. For instance, less than the aforementioned minimum amounts may be sufficient in some cases, while the upper limit specified has to be exceeded in other cases. In the case of administration of relatively large amounts, it may be advisable to divide these into several individual doses over the day.

This pharmaceutical composition will be utilized to achieve the desired pharmacological effect by preferably topical administration into the eye to a patient in need thereof, and will have advantageous properties in terms of drug release, bioavailability, and/or compliance in mammals. A patient, for the purpose of this invention, is a mammal, including a human, in need of treatment for the particular condition or disease.

The pharmaceutical composition according to the invention is chemically stable for more than 18 months, preferably more than 24 months. Chemically stable according the present invention means that the active agent does not degrade significantly (<1%) during storage.

In this connection the topical ophthalmological pharmaceutical composition according to the invention contains 4-(4-amino-3-fluorophenoxy)pyridine-2-carboxylic acid methylamide (IUPAC: 4-(4-amino-3-fluorophenoxy)-N-methylpyridine-2-carboxamide) (AFP-PMA) in an amount of equal or less than 0.05%, that means from 0.001% to a maximum of 0.05%, preferably in an amount of equal or less than 0.025%, that means from 0.001% to a maximum of 0.025%, most preferably in an amount of equal or less than 0.01%, that means from 0.001% to a maximum of 0.01% by weight based on the amount of the compound of the formula (I).

### Process for Manufacturing

Various methods can be used to prepare the ophthalmological pharmaceutical composition according to the invention. First the pharmaceutically acceptable vehicle is prepared by optionally mixing the applicable vehicle or mixture of vehicles with the pharmaceutically acceptable excipients. Thereafter the active agent is dispersed or suspended into said mixture. The process may also include sterilization e.g. by sterile precipitation, gamma irradiation, sterile filtration, heat sterilization, aseptic filling, or a combination of such optional steps.

The present invention also relates to a process for the manufacturing of a topical ophthalmological pharmaceutical composition according to the invention, wherein the compound of the present invention is suspended in an applicable pharmaceutically acceptable vehicle optionally in the presence of further one or more pharmaceutically acceptable excipients and the suspension is homogenized.

Preference is given to a process for the manufacturing of a topical ophthalmological pharmaceutical composition according to the invention, wherein
a) the applicable pharmaceutically acceptable vehicle or a mixture of applicable pharmaceutically acceptable vehicles is prepared by mixing the vehicles optionally in the presence of a further one or more pharmaceutically acceptable excipients,
b) the compound of the present invention, preferably regorafenib, more preferably regorafenib monohydrate, is suspended into said applicable pharmaceutically acceptable vehicle or mixture for example at room temperature, optionally in the presence of a further one or more pharmaceutically acceptable excipients,
c) the suspension is homogenized by stirring, shaking, vortexing or high-shear homogenization, preferably stirring, at room temperature or not more than 40° C.,
d) the suspension is subdivided into single units and filled into applicable vials, container, tube, flask, dropper and/or syringe.

Optionally in step a) the further one or more pharmaceutically acceptable excipients are added to the applicable pharmaceutically acceptable vehicle at elevated temperatures for example of 40 to 70° C.

### Method of Treating Ophthalmological Disorders

The present invention also relates to a use of the pharmaceutical composition according to the invention to treat or prevent ophthalmological disorders.

Furthermore the present invention also relates to a method for treating or preventing an ophthalmological disorder comprising administering a pharmaceutical composition containing a pharmaceutically effective amount of an active agent according to the present invention.

Examples of ophthalmological disorders according to the invention include but are not limited to age-related macular degeneration (AMD), choroidal neovascularization (CNV), choroidal neovascular membrane (CNVM), cystoid macula edema (CME), epi-retinal membrane (ERM) and macular hole, myopia-associated choroidal neovascularization, vascular streaks, retinal detachment, diabetic retinopathy, diabetic macular edema (DME), atrophic changes of the retinal pigment epithelium (RPE), hypertrophic changes of the retinal pigment epithelium (RPE), retinal vein occlusion, choroidal retinal vein occlusion, macular edema, macular edema due to retinal vein occlusion, retinitis pigmentosa, Stargardt's disease, glaucoma, inflammatory conditions of the eye such as e.g. uveitis, scleritis or endophthalmitis, cataract, refractory anomalies such as e.g. myopia, hyperopia or astigmatism and ceratoconus and retinopathy of prematurity. In addition, examples include but are not limited to angiogenesis in the front of the eye like corneal angiogenesis following e.g. keratitis, corneal transplantation or keratoplasty, corneal angiogenesis due to hypoxia (extensive contact lens wearing), pterygium conjunctivae, subretinal edema and intraretinal edema. Examples of age-related macular degeneration (AMD) include but are not limited to dry or nonexudative AMD, or wet or exudative or neovascular AMD.

Preference is given to age-related macular degeneration (AMD) like dry AMD, wet AMD or choroidal neovascularization (CNV).

Another embodiment or the present invention is a topical ophthalmological pharmaceutical composition for the treatment or prevention of a posterior eye disease wherein the composition is a suspension comprising an active agent applicable for the treatment or prevention of a posterior eye disease suspended in a applicable pharmaceutically acceptable vehicle.

Preference is given to a suspension based on a non-aqueous vehicle, more preferably to a suspension based on a hydrophobic vehicle.

Examples of posterior eye diseases include but are not limited to age-related macular degeneration (AMD), choroidal neovascularization (CNV), choroidal neovascular membrane (CNVM), cystoid macula edema (CME), epi-retinal membrane (ERM) and macular hole, myopia-associated choroidal neovascularization, vascular streaks, retinal detachment, diabetic retinopathy, diabetic macular edema (DME), atrophic changes of the retinal pigment epithelium (RPE), hypertrophic changes of the retinal pigment epithelium (RPE), retinal vein occlusion, choroidal retinal vein occlusion, macular edema, macular edema due to retinal vein occlusion, retinitis pigmentosa, Stargardt's disease and retinopathy of prematurity.

Preferred posterior eye diseases include age-related macular degeneration (AMD) like dry AMD, wet AMD or choroidal neovascularization (CNV).

Examples of age-related macular degeneration (AMD) include but are not limited to dry or nonexudative AMD, or wet or exudative or neovascular AMD.

Active agents applicable for the treatment or prevention of a posterior eye disease according to the present invention include but are not limited to signal transduction inhibitors targeting receptor kinases of the domain families of e.g. VEGFR, PDGFR, FGFR and their respective ligands or other pathway inhibitors like VEGF-Trap (aflibercept), pegaptanib, ranibizumab, pazopanib, bevasiranib, KH-902, mecamylamine, PF-04523655, E-10030, ACU-4429, volociximab, sirolismus, fenretinide, disulfiram, sonepcizumab, regorafenib, sorafenib and/or tandospirone. These agents include, by no way of limitation, antibodies such as Avastin (bevacizumab). These agents also include, by no way of limitation, small-molecule inhibitors such as STI-571/Gleevec (Zvelebil, Curr. Opin. Oncol., Endocr. Metab. Invest. Drugs 2000, 2(1), 74-82), PTK-787 (Wood et al., Cancer Res. 2000, 60(8), 2178-2189), SU-11248 (Demetri et al., Proceedings of the American Society for Clinical Oncology 2004, 23, abstract 3001), ZD-6474 (Hennequin et al., 92nd AACR Meeting, New Orleans, March 24-28, 2001, abstract 3152), AG-13736 (Herbst et al., Clin. Cancer Res. 2003, 9, 16 (suppl 1), abstract C253), KRN-951 (Taguchi et al., 95th AACR Meeting, Orlando, Fla., 2004, abstract 2575), CP-547,632 (Beebe et al., Cancer Res. 2003, 63, 7301-7309), CP-673,451 (Roberts et al., Proceedings of the American Association of Cancer Research 2004, 45, abstract 3989), CHIR-258 (Lee et al., Proceedings of the American Association of Cancer Research 2004, 45, abstract 2130), MLN-518 (Shen et al., Blood 2003, 102, 11, abstract 476), and AZD-2171 (Hennequin et al., Proceedings of the American Association of Cancer Research 2004, 45, abstract 4539), PKC412, nepafenac.

Preference is given to regorafenib, bevacizumab, aflibercept, pegaptanib, ranibizumab, pazopanib and/or bevasiranib.

Suitable pharmaceutically acceptable vehicles according to the present invention include but are not limited to oleoyl polyethyleneglycol gylcerides, linoleoyl polyethyleneglycol gylcerides, lauroyl polyethyleneglycol gylcerides, hydrocarbon vehicles like liquid paraffin (Paraffinum liquidum, mineral oil), light liquid paraffin (low viscosity paraffin, Paraffinum perliquidum, light mineral oil), soft paraffin (vaseline), hard paraffin, vegetable fatty oils like castor oil, peanut oil or sesame oil, synthetic fatty oils like middle chain trigylcerides (MCT, triglycerides with saturated fatty acids, preferably octanoic and decanoic acid), isopropyl myristate, caprylocaproyl macrogol-8 glyceride, caprylocaproyl polyoxyl-8 glycerides, wool alcohols like cetylstearylalcohols, wool fat, glycerol, propylene glycol, propylene glycol diesters of caprylic/capric acid, polyethyleneglycols (PEG) or a mixture of thereof.

Preference is given to non-aqueous pharmaceutically acceptable vehicles which include but are not limited to middle chain trigylcerides (MCT, triglycerides with saturated fatty acids, preferably octanoic and decanoic acid, isopropyl myristate, caprylocaproyl macrogol-8 glyceride, caprylocaproyl polyoxyl-8 glycerides, oleoyl polyethyleneglycol glycerides, oleoyl macrogol-6 glycerides (Labrafil M 1944 CS), linoleoyl macrogol-6 glycerides (Labrafil M2125 CS=linoleoyl polyoxyl-6 glycerides), lauroyl macrogol-6 glycerides (Labrafil M 2130 CS=lauroyl polyoxyl-6 glycerides)), hydrocarbon vehicles, fatty oils like castor oil or a mixture of thereof. Most preferably hydrophobic vehicles are used like hydrocarbon vehicles which include but are not limited to liquid paraffin or light liquid paraffin or a mixture thereof.

Very surprisingly the suspension according to the present invention comprising a lipophilic vehicle like liquid or light liquid paraffin provides by topical administration a sufficient amount of the active agent to the back of the eye which is effective for treating a posterior eye disease.

Suitable further pharmaceutically acceptable excipients used in the topical ophthalmological pharmaceutical composition according to the present invention include but are not limited to surfactants, polymer based carriers like gelling agents, organic co-solvents, pH active components, osmotic active components and preservatives.

The pharmaceutically acceptable vehicle is the basis of the topical ophthalmological pharmaceutical composition according to the present invention and is present in the composition in a minimum concentration of 75%, preferably 80%, more preferably 85% and in a maximum concentration of 99.9%, preferably 99%, more preferably 98% by weight of the total amount of the composition. The active ingredient used in the topical ophthalmological pharmaceutical composition is used preferably in a micronized form.

Micronization can be achieved by standard milling methods, preferably by air jet milling, known to a skilled person. The micronized form can have a mean particle size of from 0.5 to 10 μm, preferably from 1 to 6 μm, more preferably from 2 to 3 μm. The indicated particle size is the mean of the particle size distribution measured by laser diffraction known to a skilled person (measuring device: HELOS, Sympatec).

The concentration of the active ingredient in the pharmaceutical composition is from 0.1 to 100 mg/ml, preferably from 1 to 50 mg/ml, more preferably from 2 to 40 mg/ml.

The pharmaceutical composition according to the invention can be administered as the sole pharmaceutical composition or in combination with one or more other pharmaceutical compositions or active agents where the combination causes no unacceptable adverse effects.

“Combination” means for the purposes of the invention not only a dosage form which contains all the active agents (so-called fixed combinations), and combination packs containing the active agents separate from one another, but also active agents which are administered simultaneously or sequentially, as long as they are employed for the prophylaxis or treatment of the same disease.

Since the combination according to the invention is well tolerated and is potentially effective even in low dosages, a wide range of formulation variants is possible. Thus, one possibility is to formulate the individual active ingredients of the combination according to the invention separately. In this case, it is not absolutely necessary for the individual active ingredients to be taken at the same time; on the contrary, sequential intake may be advantageous to achieve optimal effects. It is appropriate with such separate administration to combine the formulations of the individual active ingredients simultaneously together in a suitable primary packaging. The active ingredients are present in the primary packaging in each case in separate containers which may be, for example, tubes, bottles or blister packs. Such separate packaging of the components in the joint primary packaging is also referred to as a kit.

In one embodiment, the pharmaceutical compositions of the present invention can be combined with other ophthalmological agents. Examples of such agents include but are not limited to carotenoids like lycopene, lutein, zeaxanthin, phytoene, phytofluene, carnosic acid and derivatives thereof like carnosol, 6,7-dehydrocarnosic acid, 7-ketocarnosic acid, a zink source like zinc oxide or a zinc salt like its chloride, acetate, gluconate, carbonate, sulphate, borate, nitrate or silicate salt, copper oxide, vitamin A, vitamin C, vitamin E and/or β-carotene.

In another embodiment, the pharmaceutical compositions of the present invention can be combined with other signal transduction inhibitors targeting receptor kinases of the domain families of e.g. VEGFR, PDGFR, FGFR and their respective ligands or other pathway inhibitors like VEGF-Trap (aflibercept), pegaptanib, ranibizumab, pazopanib, bevasiranib, KH-902, mecamylamine, PF-04523655, E-10030, ACU-4429, volociximab, sirolismus, fenretinide, disulfiram, sonepcizumab and/or tandospirone. These agents include, by no way of limitation, antibodies such as Avastin (bevacizumab). These agents also include, by no way of limitation, small-molecule inhibitors such as STI-571/Gleevec (Zvelebil, Curr. Opin. Oncol., Endocr. Metab. Invest. Drugs 2000, 2(1), 74-82), PTK-787 (Wood et al., Cancer Res. 2000, 60(8), 2178-2189), SU-11248 (Demetri et al., Proceedings of the American Society for Clinical Oncology 2004, 23, abstract 3001), ZD-6474 (Hennequin et al., 92nd AACR Meeting, New Orleans, Mar. 24-28, 2001, abstract 3152), AG-13736 (Herbst et al., Clin. Cancer Res. 2003, 9, 16 (suppl 1), abstract C253), KRN-951 (Taguchi et al., 95th AACR Meeting, Orlando, Fla., 2004, abstract 2575), CP-547,632 (Beebe et al., Cancer Res. 2003, 63, 7301-7309), CP-673,451 (Roberts et al., Proceedings of the American Association of Cancer Research 2004, 45, abstract 3989), CHIR-258 (Lee et al., Proceedings of the American Association of Cancer Research 2004, 45, abstract 2130), MLN-518 (Shen et al., Blood 2003, 102, 11, abstract 476), and AZD-2171 (Hennequin et al., Proceedings of the American Association of Cancer Research 2004, 45, abstract 4539), PKC412, nepafenac.

Preference is given to a combination with bevacizumab, aflibercept, pegaptanib, ranibizumab, pazopanib and/or bevasiranib.

Generally, the use of the other ophthalmological agents in combination with the pharmaceutical compositions of the present invention will serve to:

(1) yield better efficacy as compared to administration of either agent alone,

(2) provide for the administration of lesser amounts of the administered agents,

(3) provide for treating a broader spectrum of mammals, especially humans,

(4) provide for a higher response rate among treated patients,

(5) yield efficacy and tolerability results at least as good as those of the agents used alone, compared to known instances where other agent combinations produce antagonistic effects. It is believed that one skilled in the art, using the preceding information and information available in the art, can utilize the present invention to its fullest extent.

It should be apparent to one of ordinary skill in the art that changes and modifications can be made to this invention without departing from the spirit or scope of the invention as it is set forth herein.

All publications, applications and patents cited above and below are incorporated herein by reference.

The weight data are, unless stated otherwise, percentages by weight and parts are parts by weight.

### EXAMPLES

### HPLC Methods

Two separate HPLC methods were developed for the determination of regorafenib content, unidentified degradation products and unidentified secondary components, as well as for the determination of the specific degradation product 4-(4-amino-3-fluorophenoxy)pyridine-2-carboxylic acid methylamide (AFP-PMA), respectively, within pharmaceutical formulations.

1) HPLC method for the determination of regorafenib content, unidentified secondary components, and unidentified degradation products: Samples were prepared by dilution of drawn formulation aliquots with water/acetonitrile (25/75) to a final regorafenib concentration of 100 μg/ml. 10 μl of each sample were injected into an Agilent 1100 HPLC system (Agilent, Waldbronn, Germany), and samples were run on a heated (40° C.) Symmetry C18 column (150×4.6 mm-3.5 μm particle size, Waters, Eschborn, Germany) applying a flow rate of 1 ml/min. The mobile phase consisted of a mixture of potassium phosphate buffer pH 2.4 (A) and acetonitrile/ethanol (6/4) (B). The following gradient was applied: minute 0: A, 60%/B, 40%; minute 12: A, 20%/B, 80%; minute 16: A, 20%/B, 80%; minute 16.5: A, 60%/B, 40%; minute 20: A, 60%/B, 40%. Regorafenib, unidentified secondary components, and unidentified degradation products were quantified using a DAD detector at a wavelength of 265 nm. Regorafenib content (column 3 in tables below) within formulations was quantified by using an external 2-point calibration straight line. Unidentified secondary components and unidentified degradation products (columns 5-7 in tables below) are described as % of summarized sample-related peak areas. Precision of the system was determined with each sample set run, by six times injection of a 100% regorafenib standard (e.g. 100 μg/ml), coefficient of variation of peak areas resulting from these six injections was always below 2%. Relative Y-axis intercept of a 2-point (e.g. 50 μg/ml, 100 μg/ml) calibration straight line was always below 3% (referring to 100% Regorafenib standard). The regorafenib peak appears at 11.5 minutes.

2) HPLC method for the determination of the specific degradation product 4-(4-amino-3-fluorophenoxy)pyridine-2-carboxylic acid methylamide (IUPAC: 4-(4-amino-3-fluorophenoxy)-N-methylpyridine-2-carboxamide) (AFP-PMA). Samples were prepared by dilution of drawn formulation aliquots with aceton to a final regorafenib concentration of 3000 μg/ml. 15 μl of each sample were injected into an Agilent 1100 HPLC system (Agilent, Waldbronn, Germany), and samples kept at 10° in the autosampler were run on a Symmetry C18 column (150×4.6 mm-3.5 μm particle size, Waters, Eschborn, Germany) held at 20° C. with a flow rate of 1 ml/min. The mobile phase consisted of a mixture of potassium phosphate buffer pH 2.4 (A) and acetonitrile/ethanol (6/4) (B). The following gradient was applied: minute 0: A, 62%/B, 38%; minute 5: A, 44%/B, 56%; minute 5.01: A, 15%/B, 85%; minute 9: A, 15%/B, 85%; minute 9.01: A, 62%/B, 38%; minute 12: A, 62%/B, 38%. 4-(4-amino-3-fluorophenoxy)pyridine-2-carboxylic acid methylamide (column 4 in tables below) was quantified using a DAD detector at a wavelength of 232 nm, referring to an external 3-point (e.g. 0.04 μg/ml, 0.1 μg/ml, 1 μg/ml) calibration straight line. The 4-(4-amino-3-fluorophenoxy)pyridine-2-carboxylic acid methylamide peak appears at 3.9 minutes. Limit of detection (LOD) and limit of quantification (LOQ) of 4-(4-amino-3-fluorophenoxy)pyridine-2-carboxylic acid methylamide were determined for two different matrices (water and paraffin), and were: LOD: 4 ppm=0.0004% (water), 13 ppm=0.0013% (paraffin); LOQ: 13 ppm=0.0013% (water) and 43 ppm=0.0043% (paraffin).

### Example 1

### Ophthalmological Suspension Comprising Regorafenib Monohydrate in Oleoyl Polyethyleneglycol Glyceride (20 mg/ml)

200 mg of micronized regorafenib monohydrate was suspended in oleoyl polyethyleneglycol glyceride (10 ml). The suspension was homogenized by stirring at room temperature for 15 minutes.

Stability of regorafenib in oleoyl polyethyleneglycol glyceride was tested at a concentration of 3 mg/ml over 4 weeks at 25° C., 60% relative humidity (r.h.) and 40° C., 75% r.h. Regorafenib content ranged between 95.0-101% of theoretical concentration, largest unidentified degradation product ranged from 0.3 to 0.7%. 4-(4-amino-3-fluorophenoxy)pyridine-2-carboxylic acid methylamide (AFP-PMA) content was below <13 ppm=0.0013% (<LOD determined for paraffin based formulation, Table 2). For analytical details see HPLC Method section above.

TABLE 2
Content and stability of regorafenib within oleoyl polyethyleneglycol glyceride based formulation:

| | | | 4 | 5 | 6 | 7 |
|---|---|---|---|---|---|---|
| | | 3 | AFP-PMA content | Largest un- | Largest un- | Largest un- |
| | | regorafenib content | (% referring to | identified secondary | identified secondary | identified degradation |
| 1 | 2 | (% of theoretical), | Regorafenib), | component in standard | component in sample | product in sample |
| Storage | Storage | via external | via external | (% of summarized | (% of summarized | (% of summarized |
| time | condition | calibration | calibration | peak areas) | peak areas) | peak areas) |
| 0 | | 95.0 | <0.0013 | 0.04 | 0.04 | 0.7 |
| 1 day | 25° C./ | 101 | <0.0013 | 0.04 | 0.04 | 0.6 |
| | 60% r.h. | | | | | |
| 4 weeks | 25° C./ | 99.0 | <0.0013 | 0.04 | 0.04 | 0.3 |
| | 60% r.h. | | | | | |
| 4 weeks | 40° C./ | 98.7 | <0.0013 | 0.04 | 0.04 | 0.3 |
| | 75% r.h. | | | | | |

### Example 2

### Ophthalmological Suspension Comprising Regorafenib Monohydrate in Liquid Paraffin (20 mg/ml)

400 mg of micronized regorafenib monohydrate was suspended in 20 ml of light liquid paraffin. The suspension was homogenized by stirring at room temperature for 15 minutes.

Stability of the suspension was tested at a concentration of 20 mg/ml over 13 weeks at 25° C., 60% relative humidity (r.h.) and 40° C., 75% r.h. Regorafenib content ranged between 74.8-99.6% of theoretical concentration. The observed fluctuation is most likely due to inhomogeneity of the sample after manual shaking of the suspension. No unidentified degradation product was observed in chromatograms. AFP-PMA content was below <43 ppm=0.0043% (<LOQ determined for paraffin based formulation, Table 3). For analytical details see Analytics section above.

TABLE 3
Content and stability of regorafenib within paraffin based formulation.

| | | | 4 | 5 | 6 | 7 |
|---|---|---|---|---|---|---|
| | | 3 | AFP-PMA content | Largest un- | Largest un- | Largest un- |
| | | regorafenib content | (% referring to | identified secondary | identified secondary | identified degradation |
| 1 | 2 | (% of theoretical), | Regorafenib), | component in standard | component in sample | product in sample |
| Storage | Storage | via external | via external | (% of summarized | (% of summarized | (% of summarized |
| time | condition | calibration | calibration | peak areas) | peak areas) | peak areas) |
| 0 | | 99.6 | <0.0043 | 0.04 | 0.04 | — |
| 4 weeks | 25° C./ | 85.4 | <0.0043 | 0.04 | 0.04 | — |
| | 60% r.h. | | | | | |
| 4 weeks | 40° C./ | 74.8 | <0.0043 | 0.04 | 0.04 | — |
| | 75% r.h. | | | | | |
| 13 weeks | 25° C./ | 96.9 | <0.0043 | 0.04 | 0.04 | — |
| | 60% r.h. | | | | | |
| 13 weeks | 40° C./ | 94.6 | <0.0043 | 0.04 | 0.04 | — |
| | 75% r.h. | | | | | |

### Example 3

### Ophthalmological Suspension Comprising Regorafenib Monohydrate in Water Based Vehicle (20 mg/ml)

1.7 g of hydroxypropymethylcellulose 15 cp (HPMC) was dispersed in isotonic sodium chloride solution (48 g, 0.9% NaCl in water) at 70° C. The mixture was cooled down to room temperature while stirring. At room temperature evaporated water, and subsequently polysorbate 80 (0.5 g) was added and dissolved under moderate stirring. 518 mg of regorafenib monohydrate was added to an aliquot of the prepared vehicle (24.5 g) and the suspension was homogenized by gently stirring at room temperature for 15 minutes.

Stability of the suspension was tested at a concentration of 10 mg/ml over 13 weeks at 25° C., 60% relative humidity (r.h.) and 40° C., 75% r.h. Regorafenib content ranged between 103-112% of theoretical concentration. The observed fluctuation is most likely due to inhomogeneity of the sample after manual shaking of the suspension. Largest unidentified degradation product was <0.1% of summarized sample related peak areas. Amount of AFP-PMA was determined only after 9 weeks storage.

TABLE 4
Content and Stability of Regorafenib within water based formulation.

| | | | 4 | 5 | 6 | 7 |
|---|---|---|---|---|---|---|
| | | 3 | AFP-PMA content | Largest un- | Largest un- | Largest un- |
| | | regorafenib content | (% referring to | identified secondary | identified secondary | identified degradation |
| 1 | 2 | (% of theoretical), | Regorafenib), | component in standard | component in sample | product in sample |
| Storage | Storage | via external | via external | (% of summarized | (% of summarized | (% of summarized |
| time | condition | calibration | calibration | peak areas) | peak areas) | peak areas) |
| 0 | | 103 | n.d. | 0.04 | 0.04 | — |
| 4 weeks | 25° C./ | 104 | n.d. | 0.1 | 0.04 | — |
| | 60% r.h. | | | | | |
| 4 weeks | 40° C./ | 112 | n.d. | 0.1 | 0.04 | — |
| | 75% r.h. | | | | | |
| 9 weeks | 25° C./ | | 0.0056 | | | |
| | 60% r.h. | | | | | |
| 9 weeks | 40° C./ | | 0.0086 | | | |
| | 75% r.h. | | | | | |
| 13 weeks | 25° C./ | 104 | n.d. | 0.06 | 0.04 | — |
| | 60% r.h. | | | | | |
| 13 weeks | 40° C./ | 104 | n.d. | 0.06 | 0.04 | — |
| | 75% r.h. | | | | | |

In tables 2, 3 and 4 above column 5 describes the percental amount of the largest unidentified secondary component in the standard used in the HPLC method to be compared with the value of column 6 which describes the percental amount of the same unidentified secondary component in the formulation. Column 7 describes the percental amount of the largest unidentified degradation product in the formulation which is not AFP-PMA. Said degradation product is not detectable in the standard but is formed in the formulation.

### Example 4

### Ophthalmological Suspension Comprising Regorafenib Monohydrate in Middle Chain Triglycerides (MCT, Miglyol) (20 mg/ml)

Example 4 was prepared according to example 1.

TABLE 5
Content and stability of regorafenib within MCT- based formulation.

| | | | 4 | 5 | 6 | 7 |
|---|---|---|---|---|---|---|
| | | 3 | AFP-PMA content | Largest un- | Largest un- | Largest un- |
| | | regorafenib content | (% referring to | identified secondary | identified secondary | identified degradation |
| 1 | 2 | (% of theoretical), | Regorafenib), | component in standard | component in sample | product in sample |
| Storage | Storage | via external | via external | (% of summarized | (% of summarized | (% of summarized |
| time | condition | calibration | calibration | peak areas) | peak areas) | peak areas) |
| 0 | | 106.0 | <0.0043 | <0.1 | <0.1 | <0.1 |
| 4 weeks | 25° C./ | 99.5 | <0.0043 | <0.1 | <0.1 | <0.1 |
| | 60% r.h. | | | | | |
| 4 weeks | 40° C./ | 101.8 | <0.0043 | <0.1 | <0.1 | <0.1 |
| | 75% r.h. | | | | | |
| 13 weeks | 25° C./ | 101.0 | <0.0043 | <0.1 | <0.1 | <0.1 |
| | 60% r.h. | | | | | |
| 13 weeks | 40° C./ | 101.5 | <0.0043 | <0.1 | <0.1 | <0.1 |
| | 75% r.h. | | | | | |

### Example 5

### Ophthalmological Suspension Comprising Regorafenib Monohydrate in Oculentum Simplex (20 mg/g)

100 mg of micronized regorafenib monohydrate was suspended in 4900 mg oculentum simplex (composition: cholesterole 1%, liquid paraffin 42.5%, soft paraffin 56.5% by weight). The suspension was homogenized by stirring at room temperature in an Agate motar for approximately 1 minute.

### Example 6

### Topical Efficacy of Different Formulations Containing Regorafenib in the Rat Laser-Induced Choroidal Neovascularization (CNV) Model

The aim of this study was to determine whether twice daily topical administration (eye drops) of the topical ophthalmological pharmaceutical compositions according to the invention results in a decrease of vascular leakage and/or choroidal neovascularization in a rat model of laser-induced choroidal neovascularization (Dobi et al, Arch. Ophthalmol. 1989, 107(2), 264-269 or Frank et al, Curr. Eye Res. 1989 March, 8(3), 239-247)

For this purpose, a total of 133 pigmented Brown-Norway rats with no visible sign of ocular defects were selected and randomly assigned to eight groups of six to eight animals each. On day 0, the animals were anaesthetized by an intraperitoneal injection (15 mg/kg xylazine and 80 mg/kg ketamine (dissolved in water containing 5 mg/ml chlorobutanol hemihydrate and propylenglycol) After instillation of one drop of 0.5% atropin (dissolved in 0.9% saline containing Benzalkoniumchloride) to dilate the pupils, choroidal neovascularization was induced by burning six holes in the retina (disruption of Bruch's membrane) of one eye per animal (lesion size: 50 μm, laser intensity: 150 mW; stimulus duration: 100 ms) using a 532 nm argon laser.

The following formulations were included:
a) 100% oleoyl polyethyleneglycol glycerides as used in example 1 (vehicle control), n=8
b) Example 1 (20 mg/ml, suspension), n=8
c) 100% light liquid paraffin as used in example 2 (vehicle control), n=8
d) Example 2 (20 mg/ml, suspension), n=8
e) Water-based vehicle (Hydroxypropymethylcellulose 15 cp 3.5%, polysorbate 80 0.5%, isotonic NaCl solution 96% as used in example 3 (vehicle control), n=6
f) Example 3 (20 mg/ml, suspension), n=6
g) 100% Miglyol as used in example 4 (vehicle control), n=8
h) Example 4 (20 mg/ml, suspension), n=7
i) 100% oculentum simplex as used in example 5 (vehicle control), n=8
j) Example 5 (20 mg/g, suspension), n=6

Of each formulation, 10 μl were applied to the affected eye twice daily at an 10:14 hour interval during the complete observation period of 23 days. The body weight of all animals was recorded before the start and once daily during the study. An angiography was performed on day 21 using a fluorescence fundus camera (Kowe Genesis Df, Japan). Here, after anesthesia and pupillary dilation, 10% sodium fluorescein (dye, dissolved in water) was subcutaneously injected and pictures were recorded approximately 2 min after dye injection. The vascular leakage of the fluorescein on the angiograms was evaluated by three different examiners who were blinded for group allocation (examples 1 to 3 versus respective vehicle). Each lesion was scored with 0 (no leakage) to 3 (strongly stained), and a mean from all 6 lesions was used as the value for the respective animal. On day 23, animals were sacrificed and eyes were harvested and fixed in 4% paraformaldehyde solution for 1 hour at room temperature. After washing, the retina was carefully peeled, and the sclera-choroid complex was washed, blocked and stained with a FITC-isolectine B4 antibody in order to visualize the vasculature. Then, the sclera-choroids were flat-mounted and examined under a fluorescence microscope (Keyence Biozero) at 488 nm excitation wavelength. The area (in μm²) of choroidal neovascularization was measured using ImageTool software.

### Results:

### A) Efficacy Regarding Vascular Leakage (Angiography Scores Day 21):

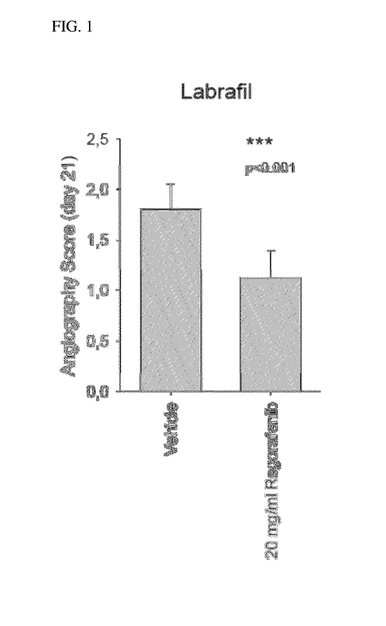

FIG. 1: Angiography scores of vehicle (oleoyl polyethyleneglycol glycerides (Labrafil), formulation a) and regorafenib (example 1, formulation b) treated animals at day 21. Data are presented as mean±SD, p-value according to t-test. N=8 per group.

TABLE 6
Raw data of the histogram depicted in FIG. 1. Single
values represent the means from three different
observers blinded with respect to treatment.

| | 100% oleoyl polyethyleneglycol | Example 1 |
|---|---|---|
| Animal | glycerides (formulation a) | (formulation b) |
| 1 | 1.80 | 1.14 |
| 2 | 1.72 | 0.67 |
| 3 | 1.63 | 1.44 |
| 4 | 1.72 | 0.90 |
| 5 | 1.67 | 1.00 |
| 6 | 2.00 | 1.22 |
| 7 | 1.56 | 1.33 |
| 8 | 2.33 | 1.33 |

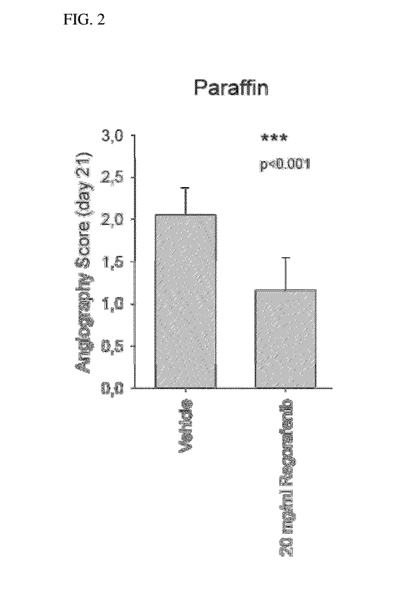

FIG. 2: Angiography scores of vehicle (paraffin, formulation c) and regorafenib (example 2, formulation d) treated animals at day 21. Data are presented as mean±SD, p-value according to t-test. N=8 per group.

TABLE 7
Raw data of the histogram depicted in FIG. 2. Single
values represent the means from three different
observers blinded with respect to treatment.

| | 100% paraffin | Example 2 |
|---|---|---|
| Animal | (formulation c) | (formulation d) |
| 1 | 2.33 | 1.29 |
| 2 | 1.77 | 1.78 |
| 3 | 1.50 | 0.69 |
| 4 | 1.91 | 1.34 |
| 5 | 2.21 | 0.67 |
| 6 | 2.06 | 1.00 |
| 7 | 2.10 | 0.96 |
| 8 | 2.54 | 1.51 |

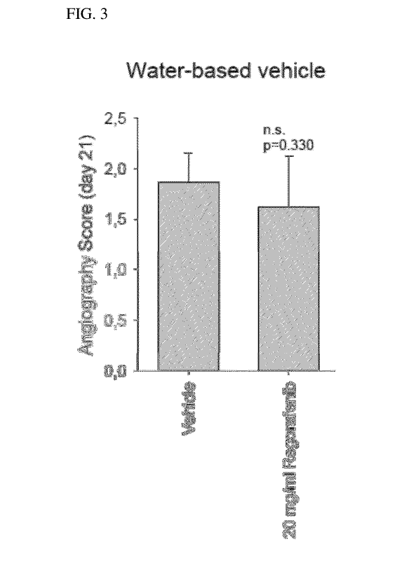

FIG. 3: Angiography scores of vehicle (water based, formulation e) and regorafenib (example 3, formulation f) treated animals at day 21. Data are presented as mean±SD, p-value according to t-test. N=6 per group.

TABLE 8
Raw data of the histogram depicted in FIG. 3. Single
values represent the means from three different
observers blinded with respect to treatment.

| Animal | formulation e | Example 3 (formulation f) |
|---|---|---|
| 1 | 1.61 | 1.78 |
| 2 | 1.78 | 1.60 |
| 3 | 1.93 | 1.34 |
| 4 | 2.27 | 2.00 |
| 5 | 1.49 | 0.80 |
| 6 | 2.10 | 2.20 |

### B) Efficacy Regarding Neovascularization (Neovascular Area Day 23):

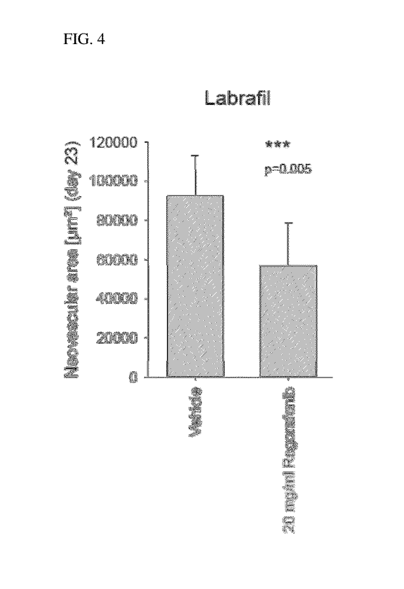

FIG. 4: Neovascular area of vehicle (oleoyl polyethyleneglycol glycerides (Labrafil), formulation a) and regorafenib (example 1, formulation b) treated animals at day 23. Data are presented as mean±SD, p-value according to t-test. N=8 per group.

TABLE 9
Raw data of the histogram depicted in FIG. 4. Single
values represent the means from all six lesions.

| | 100% oleoyl polyethyleneglycol | Example 1 |
|---|---|---|
| Animal | glycerides (formulation a) | (formulation b) |
| 1 | 134507 | 90562 |
| 2 | 70878 | 59819 |
| 3 | 84254 | 67222 |
| 4 | 86071 | 72584 |
| 5 | 93586 | 30455 |
| 6 | 69696 | 47866 |
| 7 | 103307 | 23991 |
| 8 | 98472 | 63033 |

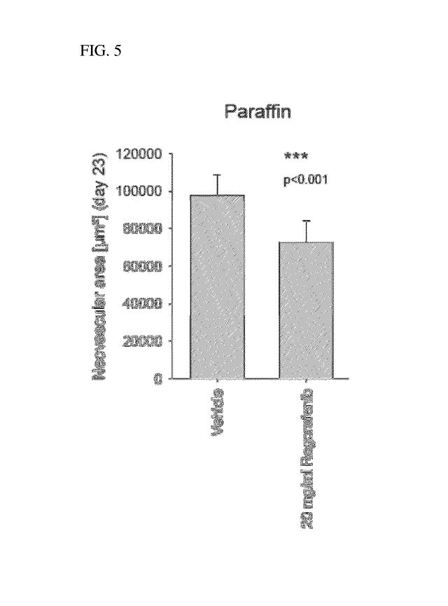

FIG. 5: Neovascular area of vehicle (paraffin, formulation c) and regorafenib (example 2, formulation d) treated animals at day 23. Data are presented as mean±SD, p-value according to t-test. N=8 per group.

TABLE 10
Raw data of the histogram depicted in FIG. 5. Single
values represent the means from all six lesions.

| | 100% paraffin | Example 2 |
|---|---|---|
| Animal | (formulation c) | (formulation d) |
| 1 | 105910 | 62047 |
| 2 | 81060 | 70927 |
| 3 | 98735 | 84481 |
| 4 | 85019 | 80151 |
| 5 | 98071 | 70568 |
| 6 | 101668 | 59804 |
| 7 | 99413 | 63145 |
| 8 | 113797 | 91466 |

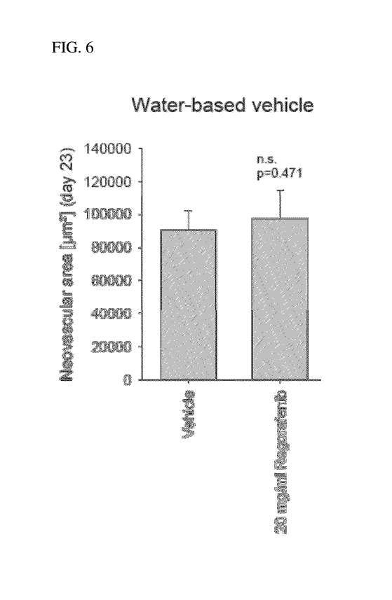

FIG. 6: Neovascular area of vehicle (water based, formulation e)) and regorafenib (example 3, formulation f) treated animals at day 23. Data are presented as mean±SD, p-value according to t-test. N=5 per group.

TABLE 11
Raw data of the histogram depicted in FIG. 6. Single
values represent the means from all six lesions.

| Animal | formulation e | Example 3 (formulation f) |
|---|---|---|
| 1 | 78759 | 107547 |
| 2 | 83420 | 117379 |
| 3 | 96239 | 72404 |
| 4 | 107654 | 99371 |
| 5 | 87960 | 91977 |

In both groups, one flatmount preparation each could not be scored due to poor tissue quality.

### Results for Example 1:

TABLE 12
(n = 8 per group)

| | | B) Choroidal |
|---|---|---|
| | A) Vascular leakage | neovascularization |
| Formulation | [angiography score] | lesion size [μm²] |
| a) 100% oleoyl | 1.80 ± 0.25 | 92596 ± 20754 |
| polyethyleneglycol | | |
| glycerides (vehicle control) | | |
| b) Regorafenib (20 mg/ml) | 1.13 ± 0.26 | 56942 ± 22025 |
| suspension in 100% oleoyl | | |
| polyethyleneglycol glycerides | | |
| (example 1) | | |
| p-value | <0.001 | 0.005 |

### Results for Example 2:

TABLE 13
(n = 8 per group)

| | | B) Choroidal |
|---|---|---|
| | A) Vascular leakage | neovascularization |
| Formulation | [angiography score] | lesion size [μm²] |
| c) 100% liquid paraffin | 2.05 ± 0.33 | 97959 ± 10580 |
| (vehicle control) | | |
| d) Regorafenib (20 mg/ml) | 1.16 ± 0.39 | 72824 ± 11496 |
| suspension in 100% liquid | | |
| paraffin (example 2) | | |
| p-value | <0.001 | <0.001 |

### Results for Example 3:

TABLE 14
(n = 6 per group for leakage, n =
5 per group for neovascularization)

| | | B) Choroidal |
|---|---|---|
| | A) Vascular leakage | neovascularization |
| Formulation | [angiography score] | lesion size [μm²] |
| e) Water-based | 1.86 ± 0.30 | 90806 ± 11414 |
| vehicle control | | |
| f) Regorafenib (20 mg/ml) | 1.62 ± 0.50 | 97736 ± 17027 |
| suspension in water-based | | |
| vehicle (example 6) | | |
| p-value | 0.330 (n.s.) | 0.471 (n.s.) |

### Results for Example 4:

TABLE 15
(n = 8 fo vehicle, n = 7 for regorafenib)

| | | B) Choroidal |
|---|---|---|
| | A) Vascular leakage | neovascularization |
| Formulation | [angiography score] | lesion size [μm²] |
| g) 100% middle chain | 1.53 ± 0.50 | 84971 ± 14882 |
| triglycerides (Miglyol, | | |
| vehicle control) | | |
| h) Regorafenib (20 mg/ml) | 1.40 ± 0.27 | 68127 ± 8954 |
| suspension in miglyol | | |
| p-value | 0.567 (n.s.) | 0.022 |

### Results for Example 5:

TABLE 16
(n = 8 for vehicle, n = 6 for regorafenib)

| | | B) Choroidal |
|---|---|---|
| | A) Vascular leakage | neovascularization |
| Formulation | [angiography score] | lesion size [μm²] |
| i) 100% Oculentum simplex | 1.41 ± 0.41 | 83301 ± 9729 |
| (vehicle control) | | |
| j) Regorafenib (20 mg/g) | 1.11 ± 0.36 | 60628 ± 17812 |
| suspension in oculentum | | |
| simplex | | |
| p-value | 0.180 (n.s.) | 0.010 |

### Example 7

### Ocular Pharmacokinetics

### A)

At day of experiment a defined dose of the test compound (regorafenib monohydrate 20 mg/ml) as suspension in different vehicles is applied to each eye by the use of an Eppendorf pipet. In a period of 24 to 96 hours after application a sequence (8-12 time points) of animals were sacrificed to get the eyes of these animals (rats). These eyes were rinsed in 1 ml of physiological saline solution at least 2 times and afterwards dried with a paper flies. To determine the total concentration of the test compound in the eye it is homogenized within a defined amount of saline solution and an aliquot of the homogenate is spiked with Acetonitrile to precipitate proteins in the solution. After centrifugation, in the supernatant the test compound and its possible known decomposition products were quantified with appropriate LC/MS-MS methods. Are the concentrations of the test compound or its possible known decomposition products to be determined in some defined compartments of the eye, the eyes are dissected into the appropriate compartments and each compartment is homogenized, handled and measured as described above.

In that way a concentration-time curve is determined; this is then used to calculate standard pharmacokinetic parameters to assess the qualification of a certain formulation (concentration maximum and half-life). The calculated standard pharmacokinetic parameters of the test compound or of the hereof released active pharmaceutical ingredient are: AUCₙₒᵣₘ, Cₘₐₓ, and MRT (mean residence time).

Pharmacokinetic parameters regarding regorafenib calculated from eye concentration-time curves for equal doses but with different formulations are shown in the table below.

TABLE 17

| formulation | Cₘₐₓ [mg/L] | AUCₙₒᵣₘ [kg*h/L] | MRT [h] |
|---|---|---|---|
| b) Labrafil (example 1) | 1.0 | 23 | 40 |
| d) Paraffin (example 2) | 1.4 | 28 | 41 |
| f) Water (example 3) | 1.8 | 5.3 | 28 |
| h) Miglyol (example 4) | 1.1 | 12 | 30 |
| j) Oculentum Simplex | 1.7 | 4.8 | 28 |
| (example 5) | | | |

### B)

Three unanaesthetized female rabbits were administered with a defined amount (30 μL) of suspension in Paraffin in the lower lacrimal sac of each eye. Using a glass capillary over a period of 60 min, several weight controlled samples (n=8) of tear fluid were collected. The determination of the concentration of the compound in the fluid and the evaluation of the pharmacokinetic parameters is the same as described above.

TABLE 18

| formulation | Cₘₐₓ [mg/L] | AUCₙₒᵣₘ [kg*h/L] | MRT [h] |
|---|---|---|---|
| d) Paraffin (example 2) | 149 | 99 | 0.6 |

The results show a surprisingly high residence time of the active agent in the tear fluid and on the cornea.

### Example 8

### Topical Efficacy of Different Formulations Containing Regorafenib in the Non-Human Primate Laser-Induced Choroidal Neovascularization (CNV) Model

The aim of this study was to determine whether twice daily topical administration (eye drops) of the topical ophthalmological pharmaceutical compositions according to the invention results in a decrease of clinically meaningful grade IV lesions in a non-human primate model of laser-induced choroidal neovascularization (Ryan, 1982, Krzystolik et al., 2002, Nork et al., 2011)

For this purpose, a total of 51 pigmented non-human primates (macaca fascicularis) with no visible sign of ocular defects were selected and randomly assigned to three groups. On day 0, the animals were anaesthetized by an intraperitoneal injection (10-12 mg/kg ketamine). After instillation of one drop of 1% tropicamide to dilate the pupils, choroidal neovascularization was induced by burning nine holes in the retina (disruption of Bruch's membrane, only in one animal, only eight lesions could be placed, animal 5 in the regorafenib/paraffin group) of one eye per animal (lesion size: 50 μm, laser intensity: 300-500 mW; stimulus duration: 50 ms) using a 532 nm argon laser. The following formulations were included:
k) light liquid paraffin or light liquid paraffin containing 2% (w/v) of hydrophobic silica (vehicle control), n=23
l) Regorafenib monohydrate 20 mg/ml suspension in light liquid paraffin, n=16
m) Regorafenib monohydrate 20 mg/ml suspension in light liquid paraffin containing 2% (w/v) of hydrophobic silica, n=12

Of each formulation, 50 μl were applied to the affected eye twice daily at an 12:12 hour interval during the complete observation period of 21 days. The body weight of all animals was recorded before the start and once daily during the study. An angiography was performed on day 21 using a fluorescence fundus camera (Kowa VX-10i, Japan or FF450 plus IRu Retina Camera, Carl Zeiss Meditec, Jena, Germany). Here, after anesthesia and pupillary dilation, 10% sodium fluorescein (dye, dissolved in water, 0.1 mL/kg) was subcutaneously injected and pictures were recorded approximately 2 min (early phase) and 10 min (late phase) after dye injection. The vascular leakage of the fluorescein on the angiograms was scored by an examiner who was blinded for group allocation according to the following grades: with “grade I” (no hyperfluorescence), “grade II” (hyperfluorescence without dye leakage), “grade III” (early hyperfluorescence and late mild dye leakage) or “grade IV” (early hyperfluorescence and late severe dye leakage beyond the borders of the burn area). Grade IV lesions are considered clinically meaningful and were thus considered as primary readout. Per animal, the percentage of grade IV lesions out of the nine lesions was taken as raw data.

### Formulation k):

Light liquid paraffin was filled in brown glass bottles closed with rubber stoppers and sealed with a flanged aluminum closure. Eleven of 23 bottles were terminally sterilized using gamma-radiation at not less than 25 kGY.

Optionally 5.0 g hydrophobic colloidal silica (Aerosil R972) was added in 206.5632 g light liquid paraffin and homogenized for 45 minutes at using a batch high shear mixer. The medium was filled in brown glass bottles closed with rubber stoppers and sealed with a flanged aluminum closure.

### Formulation l):

41.494 g of micronized regorafenib monohydrate (equivalent to 40.0 g regorafenib) was suspended in 1676.50 g of light liquid paraffin. The suspension was stirred at room temperature for 10 minutes. Afterwards, the suspension was homogenized for 30 minutes at 10,000 to 12,000 rpm using an inline high shear mixer. Temperature was controlled at not more than 40° C. The suspension was filled into brown glass bottles during continuous stirring and the bottles were closed with rubber stoppers and sealed with a flanged aluminum closure. Terminal sterilization of eleven of 16 bottles were performed by gamma-irradiation at not less than 25 kGy.

### Formulation m):

5.0 g hydrophobic colloidal silica (Aerosil R972) was added in 206.563 g light liquid paraffin and homogenized for 45 minutes at using a batch high shear mixer. Temperature was set at not more than 40° C. 5.187 g regorafenib monohydrate (equivalent to 5.0 g regorafenib) was suspended in the dispersion medium and the suspension was homogenized for 45 minutes at 10,000 to 12,000 rpm using a batch high shear mixer. Temperature was controlled at not more than 40° C. The suspension was filled into brown glass bottles during continuous stirring and the bottles were closed with rubber stoppers and sealed with a flanged aluminum closure. Terminal sterilization of five of twelve bottles were performed by gamma-irradiation at not less than 25 kGy.

### Results:

### A) Efficacy Regarding Vascular Leakage (Angiography Scores Day 21):

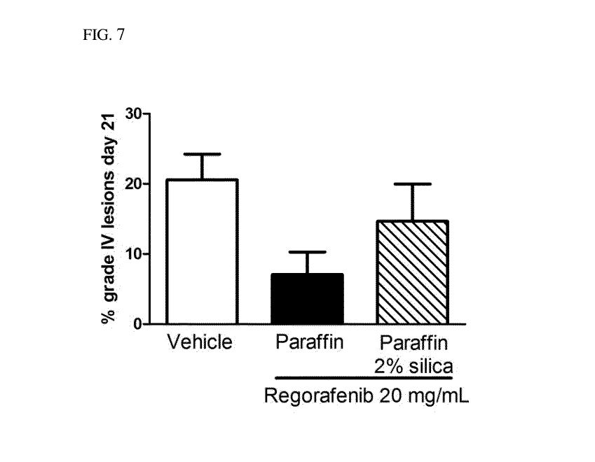

FIG. 7: % grade IV lesions in vehicle-treated animals with or without 2% hydrophobic silica (Vehicle=formulation k), and regorafenib-treated animals with or without 2% hydrophobic silica, respectively (Paraffin=formulations 1 and Paraffin 2% silica=formulation m) at day 21. Data are presented as mean±SEM.

TABLE 19
Raw data of the histogram depicted in FIG. 7. Single values
represent the % of grade IV lesions for each animal.

| | | | Regorafenib monohydrate |
|---|---|---|---|
| | | Regorafenib | 20 mg/ml Paraffin |
| | | monohydrate | containing 2% (w/v) |
| | Vehicle | 20 mg/ml paraffin | hydrophobic silica |
| Animal | (formulation k) | (formulation 1) | (formulation m) |
| 1 | 0 | 0 | 22 |
| 2 | 55 | 0 | 33 |
| 3 | 33 | 0 | 0 |
| 4 | 11 | 11 | 0 |
| 5 | 33 | 25 | 55 |
| 6 | 0 | 11 | 0 |
| 7 | 0 | 0 | 0 |
| 8 | 44 | 0 | 0 |
| 9 | 0 | 22 | 22 |
| 10 | 44 | 44 | 11 |
| 11 | 22 | 0 | 0 |
| 12 | 33 | 0 | 33 |
| 13 | 44 | 0 | |
| 14 | 0 | 0 | |
| 15 | 11 | 0 | |
| 16 | 0 | 0 | |
| 17 | 22 | | |
| 18 | 11 | | |
| 19 | 22 | | |
| 20 | 33 | | |
| 21 | 22 | | |
| 22 | 33 | | |
| 23 | 0 | | |

Although the invention has been disclosed with reference to specific embodiments, it is apparent that other embodiments and variations of the invention may be devised by others skilled in the art without departing from the true spirit and scope of the invention. The claims are intended to be construed to include all such embodiments and equivalent variations.

1. A topical ophthalmological pharmaceutical composition comprising regorafenib, a hydrate, solvate or pharmaceutically acceptable salt of regorafenib, or a polymorph thereof as active agent and at least one pharmaceutically acceptable vehicle wherein the composition is a suspension comprising the active agent suspended in the applicable pharmaceutically acceptable vehicle and wherein the composition does not contain hydrophobic silica.

2. The pharmaceutical composition of claim 1 wherein the composition does not contain any stabilizer including colloidal silica, hydrophilic or hydrophobic silicas.

3. The pharmaceutical composition of claim 1 containing regorafenib monohydrate as active agent.

4. The pharmaceutical composition of claim 1 wherein the active agent is in a solid form.

5. The pharmaceutical composition of claim 1 wherein the active agent is in a crystalline form.

6. The pharmaceutical composition of claim 1 wherein the active agent is in a microcrystalline form.

7. The pharmaceutical composition of claim 1 wherein the concentration of the active agent in the pharmaceutical composition is from 0.01 to 10% by weight of the total amount of the composition.

8. The pharmaceutical composition of claim 1 wherein the pharmaceutically acceptable vehicle is selected from the group comprising oleoyl polyethyleneglycol gylcerides, linoleoyl polyethyleneglycol gylcerides, lauroyl polyethyleneglycol gylcerides, liquid paraffin, light liquid paraffin, soft paraffin (vaseline), hard paraffin, castor oil, peanut oil, sesame oil, middle chain trigylcerides, cetylstearylalcohols, wool fat, glycerol, propylene glycol, polyethyleneglycols (PEG) or a mixture of those, water or a mixture thereof.

9. The pharmaceutical composition of claim 1 based on a non-aqueous vehicle.

10. The pharmaceutical composition of any claim 1 based on a hydrophobic vehicle.

11. The pharmaceutical composition of claim 1 wherein the pharmaceutically acceptable vehicle is selected from the group comprising liquid paraffin, light liquid paraffin or a mixture thereof.

12. The pharmaceutical composition of claim 1 comprising further pharmaceutically acceptable excipients like surfactants, polymer base carriers like gelling agents, organic co-solvents, pH active components, osmotic active components and preservatives.

13. A process for manufacturing a pharmaceutical composition according to claim 1 wherein the active agent is suspended in an applicable pharmaceutically acceptable vehicle optionally in the presence of further one or more pharmaceutically acceptable excipients and the suspension is homogenized.

14. The pharmaceutical composition of claim 1 for the use of treating or preventing an ophthalmological disorder selected from the group comprising age-related macular degeneration (AMD), choroidal neovascularization (CNV), choroidal neovascular membrane (CNVM), cystoid macula edema (CME), epi-retinal membrane (ERM) and macular hole, myopia-associated choroidal neovascularization, vascular streaks, retinal detachment, diabetic retinopathy, diabetic macular edema (DME), atrophic changes of the retinal pigment epithelium (RPE), hypertrophic changes of the retinal pigment epithelium (RPE), retinal vein occlusion, choroidal retinal vein occlusion, macular edema, macular edema due to retinal vein occlusion, retinitis pigmentosa, Stargardt's disease, glaucoma, inflammatory conditions, cataract, refractory anomalies, ceratoconus, retinopathy of prematurity, angiogenesis in the front of the eye, corneal angiogenesis following keratitis, corneal transplantation or keratoplasty, corneal angiogenesis due to hypoxia (extensive contact lens wearing), pterygium conjunctivae, subretinal edema and intraretinal edema.

15. The pharmaceutical composition of claim 14 for the use of treating or preventing an ophthalmological disorder selected from the group comprising dry AMD, wet AMD or choroidal neovascularization (CNV).

16. Method for treating or preventing an ophthalmological disorder selected from the group comprising age-related macular degeneration (AMD), choroidal neovascularization (CNV), choroidal neovascular membrane (CNVM), cystoid macula edema (CME), epi-retinal membrane (ERM) and macular hole, myopia-associated choroidal neovascularization, vascular streaks, retinal detachment, diabetic retinopathy, diabetic macular edema (DME), atrophic changes of the retinal pigment epithelium (RPE), hypertrophic changes of the retinal pigment epithelium (RPE), retinal vein occlusion, choroidal retinal vein occlusion, macular edema, macular edema due to retinal vein occlusion, retinitis pigmentosa, Stargardt's disease, glaucoma, inflammatory conditions, cataract, refractory anomalies, ceratoconus, retinopathy of prematurity, angiogenesis in the front of the eye, corneal angiogenesis following keratitis, corneal transplantation or keratoplasty, corneal angiogenesis due to hypoxia (extensive contact lens wearing), pterygium conjunctivae, subretinal edema and intraretinal edema comprising administering a pharmaceutical composition according to claim 1 containing a pharmaceutically effective amount of the active agent.

17. A topical ophthalmological pharmaceutical composition for the use of treating or preventing a posterior eye disease wherein the composition is a suspension comprising an active agent applicable for the treatment or prevention of a posterior eye disease suspended in a applicable pharmaceutically acceptable vehicle wherein the composition does not contain hydrophobic silica.

18. The topical ophthalmological pharmaceutical composition of claim 17 wherein composition does not contain any stabilizer including colloidal silica, hydrophilic or hydrophobic silicas.

19. The topical ophthalmological pharmaceutical composition of claim 17 wherein the pharmaceutically acceptable vehicle is a non-aqueous vehicle.

20. The topical ophthalmological pharmaceutical composition of claim 19 wherein the pharmaceutically acceptable vehicle is a hydrophobic vehicle.

21. The topical ophthalmological pharmaceutical composition of claim 20 wherein the pharmaceutically acceptable vehicle is selected from the group comprising liquid paraffin, light liquid paraffin or a mixture thereof.
